(19) United States
(12) Patent Application Publication
(10) Pub. No.: US 2003/0127091 A1
(43) Pub. Date: Jul. 10, 2003

(54) Scientific respiration for self-health-care

(76) Inventor: Yung Chi Chang, Arlington, TX (US)

Correspondence Address: Yung Chi Chang, #216 Volunteer Drive, Arlington, TX, 76014, US

(21) Appl. No.: 10/298,956

(22) Filed: Nov. 18, 2002

Related U.S. Application Data
(63) Continuation of application No. 09/473,193, filed on Dec. 15, 1999, now abandoned.

(51) Int. Cl. A61M 16/00

(52) U.S. Cl. 128/200.24

(57) ABSTRACT

I. Discovery of the Right Respiratory Science.

Owing to the discovery of “Haldane effect”, we comprise this Invention. This effect says, “The deoxygenation of the blood increases its ability to carry CO₂.”. So that we understand why most people are by exercise to promote health, it is with big muscles-contraction to have the “Deoxygenation of the Blood” as in exercise by walking, jogging or running to consume O₂ 5-15 times more than sitting in quiet, but few people know why and how is the right way. Especially as we see, a lot of professional athletes usually early died of heart diseases, because there is not the right respiratory science and process until today.

II. The Right Respiratory Science and Process.

We cite the right respiratory theories from the right related science as: “Haldane effect”, “Gas Diffusion Law” and the “Relations between Respiration and Autonomic Nervous Functions”. In addition to promote health, life and welfare for common people, it is to prevent from and control of many traditional functional diseases standing as rheumatism, cold and heart diseases, etc. The invented respiratory process is that, exhaling be long and slow; Inhaling be short and shallow once no longer than 1 second, by elasticity of breathing organs after exhaling; in case for more O₂ required as in sport, it just speeds the breathing rate and keeps other breathing technics the same.

### a. SPECIAL CON-APPLICATION

This is a Continuation Application, has been granted the special status. The Original application Ser No. is Ser. No. 09/473,193, and Filing Date Dec. 15, 1999.

### I. PREAMBLE

### NATURE OF THE INVENTION

b. Process:—

This Invention has been designed as a process to improve traditional Natural Respiration from nature into science in order to prevent from and control of traditional functional diseases, to promote health, life and ability for people's better welfare.

c. No Flowchart:—

This is a respiratory process of inhaling and exhaling. There is no flowchart to show the procedure of this Invention as there are in other common regular invention-applications.

### INTENDED USE OF THE INVENTION

This Invention is to start the right way, by scientific self-health-care to prevent from and control of traditional functional diseases that medication has failed, as it can control just the symptoms only until today.

Inventor:—This is a U.S. citizen retired from a breath-physical therapist registered in Texas with license #0478.

### II. CROSS-REFERENCES TO RELATED APPLICATIONS

1. A Special Application:

Based on MPEP 708.02, this Application has been granted the special status by the Board of PTO on Jun. 12, 2002, because the applicant's age has been over 65 and because the use of this Invention is related to the public safety.

2. Federally Sponsored Research:—None.

3. Original Applications:

a. 1st application Ser. No.:—06/539,660

Filing Date:—Dec. 10, 1984

Title:—Scientific Health-Breath Process

b. 2nd application Ser. No. 08/308,986

Filing Date:—Sep. 20, 1994

Title:—Scientific Respiration for Self-Health-Care

c. 3rd Continuation application Ser. No.:—09/473,193

Filing Date:—Dec. 15, 1999

Title:—As above

d. 4th Continuation application Ser. No.—10/298,956

Filing Date:—Nov. 18, 2002

Title:—As above

### III. BRIEF SUMMARY OF THE INVENTION

This Invention is comprised as the respiratory process used for normal breath to improve traditional Natural Respiration. From the cited scientific theories, we discovered that our traditional Natural Respiration has been against science to cause a lot of diseases unknowingly as:

1. Hypercapnia Standing as Rheumatism:

It is due to too much CO₂ delayed in transfer and too much HbO₂ existing in the blood over the requirement of metabolism to cause the deduction of blood-acid buffer that it is against the theory of “Haldane effect”.

2. Lower CO₂-Concentration in Trachea to Cause Diseases Standing as Cold:—

It is due to breathe too forcible or inhale too long or deep against the theory of “Gas Diffusion Law”. Another way is that as cold weather promotes the O₂ absorbed to deduct CO₂-transfer against “Haldane effect”, it is easy to cause cold also.

3. Unbalance of Autonomic Nervous Functions to Cause Diseases Standing as Heart-Disease Popular as the 1st Killer in this Society.

We create respiratory process to improve traditional Natural Respiration from nature into science as:—Exhaling be long and slow, inhaling be short and shallow by elasticity of breathing organs after exhaling.

### IV. WITHOUT REGULAR DRAWING:—AS REASON BELOW

1. Every user of the Invention has had the same breathing organs, and can manipulate and use the same organs to breathe since he was borne.

2. In original Application, the regular drawing was cancelled as in original prosecution.

3. This Invention is based on the cited scientific theories, as long as any one understood the theories, he also knows how to conduct this Invention.

### V. DETAILED DESCRIPTION OF THE INVENTION

1. Why to Have This Invention:

a. Owing to the discovery of “Haldane effect”:—

Let's cite from P.75 of Dr. West's Respiratory Physiology as “The fact that the deoxygenation of the blood increases its ability to carry CO₂ is often known as the Haldane effect”. From this effect, we discovered that in our life, a lot of functional diseases caused unknowingly are due to our Natural Respiration unscientific:

b. Against “Haldane effect” and “Gas Diffusion Law”:—As to breathe too much respiratory volume even in quiet, it is to have too much O₂ absorbed over the requirement of immediate metabolism so to delay the transfer of a end to reduce the buffer of blood-acid to cause common diseases standing as rheumatism and cold.

c. Against “Relations Between Respiration and Autonomic Nervous Functions”:—

As trying to enforce inhalation long and deep, it is to cause too much sympathetic so to have unbalance of autonomic nervous functions in various important organs, the easiest victim is the heart as many heart diseases in the society.

We create the process by science in order to correct the above deffects so to improve the Natural Respiration as the following:

2. How to Make this Invention:—As the Respiratory Process Below:

a. Exhaling is stressed long and slow to limit O₂ absorbed just for the immediate requirement of metabolism, so to avoid too much sympathetic stimulation.

b. Inhaling is limited once no longer than 1 second, and is by elasticity of respiratory organs following exhaling.

3. How to Use this Invention:—

This Invention is fixed. But in our physical conditions, the O₂ consumption is variable so that the main problem in our respiration is the O₂ supply as:

a. How to Avoid O₂ Insufficient:—

It is by speeding the breathing rate or ventilation to provide higher O₂ as in sport or exercise. By “Haldane effect”, the PO₂ in the natural air is too high to fit quiet breath, so that by this Invention to improve Natural Respiration is necessary.

b. How to Deduct too much O₂Supply:—

It is by large muscles-contraction to consume more HbO₂ as in sport with legs carrying body-weight. Also, as by exhalation with diaphragm down to limit O₂-supply and to promote abdominal pressure in assistance of heart-functions.

c. How About Inhalation:—

No matter how much volume inhaled in this Invention, is by natural inhalation. We just increase the breathing rate as in sport for more O₂ required. Other breathing technics are kept the same as in quiet.

4. In Reference to Science:

a. Owing to the discovery of “Haldane effect”, we trust that “the deoxygenationn of the blood increases its ability to carry CO₂” (for CO₂ transfer and buffer).

b. In reference of Comroe's Physiology of Respiration, our standard O₂ consumption is 26 torr as in quiet status, but natural satuation of HbO₂ is always 100 torr out of the natural PO₂-159.10 torr (20.93% of the total pressure 760 torr). It shows, in order to maintain health, the best way is to have this Invention.

5. Comparison of Natural Respiration with this Invention:

a. Defects of Natural Respiration:—Also as Merits More of this Invention plus the next item b. Stated Merits.

(1) The ability reflecting the respiratory control is limited to the impulse of O₂ required only, as by the control of the Respiratory Center in the body.

(2) Less CO₂-transfer or insufficient buffer of blood-acid, is caused due to extra O₂ inhaled over the immediate metabolic requirement.

(3) Cause various functional diseases as the above statement.

b. Merits of this Invention:—Also as Defects More of Natural Respiration plus as the above item 5.a. statement.

(1) Bring people scientific guidance to correct Natural Respiration.

(2) Deduct superstitious expense caused due to Natural Respiration against science as in superstition of religion, individuals and society.

(3) Prevent and control related functional diseases economically without medication.

(4) Provide higher ability and endurance for people in work by this Invention so to promote welfare of the people.

(5) Be as threshold to prevent and control all functional diseases by scientific self-health-care instead of medication in the near future. VI. CONCLUSION

This is to remind the Judge of PTO why we must have this Invention.

1. Discovery of “Haldane effect”:—

Owing to the discovery of the neglected “Haldane effect”, we found that our people have been caused many traditional functional diseases due to neglect the important breathing science as in daily quiet respiration to stress on large breathing volume to have too much O₂ out of the immediate metabolic requirement, so to deduct CO₂-transfer and buffer in the body, and cause various functional diseases out of control unknowingly.

2. About this Invention:—

The main use of this Invention is for quiet breath to have less O₂-supply also for sport by speeding the breathing rate to provide higher O₂-supply as required By this Invention, we still have the basic natural breathing control of the respiratory center in the body, but also we have the man-made scientific guidance to follow.

3. Busy Medical Dr.s Kept:—

As in lack of this Invention until today, people mistake traditional Natural Respiration as scientific, and misbelieve respiratory patients' medical guidance as the right respiratory science, so to have people often been caused with traditional functional diseases. This is why medical Dr.s are always busy to deal with too many patients every day even in the scientific society.

4. Reminding the Judge Once More:

In the world, nothing is more important than people's respiration. However, it is still by nature without science to cause many functional diseases until today even in the scientific age.

Our Invention is not for any enterprise to manufacture merchandise, but really for people's health and welfare. Our ancestor has set the famous word “We must do everything for the people, of the people and by the people.” This is why, we have to remind the Judge once and more.

1. My name is Yung Chi Chang, I solemnly declare that I have discovered the respiratory scientific theories and found, traditional natural breath has been against science to cause diseases as cold, rheumatism and heart diseases etc., so to create the scientific respiratory process to improve traditional natural breath as Scientific Respiration for Self-Health-Care without medicine to prevent from and control of the related diseases as to comprise the claim as:
1. exhaling long and slow by “Haldane effect” to absorb O₂ limited just fit for requirement of immediate metabolism in quiet conditions,
2. inhaling by “Gas Diffusion Law”, to have short inhalation once no longer then one second and by elasticity of breathing organs following exhaling, to limit absorbed as above, and to limit the sympathetic stimulation by “Relations of Autonomic Nervous Functions with Respiration”, so to improve traditional Natural Respiration from long and deep, and from equal inhaling and exhaling, as in case for more O₂ required, it is to speed the breathing rate and keep other breathing technics the same as above, that every one is welcome to use this Invention for self-health-care; if some one used but transfered this Invention for other's health-care, it is infrangement of the patent.
